(19) United States
(12) Patent Application Publication
(10) Pub. No.: US 2007/0207460 A1
(43) Pub. Date: Sep. 6, 2007

(54) Method For Enriching And Stabilising Components Which Contain Dna And Which Are Made Of Biological Materials

(75) Inventors: Rolf Wambutt, Berlin (DE); Frank Schubert, Berlin (DE)

Correspondence Address: COHEN, PONTANI, LIEBERMAN & PAVANE, 551 FIFTH AVENUE, SUITE 1210, NEW YORK, NY, 10176, US

(73) Assignee: Agowa Gesellschaft Fur Molekularbiologische Technologie Mbh, Berlin (DE)

(21) Appl. No.: 10/584,376

(22) PCT Filed: Dec. 7, 2004
(86) PCT No.: PCT/EP04/14015

§ 371 Date: Sep. 11, 2006

(30) Foreign Application Priority Data
Dec. 24, 2003 (DE) 103 60 968.7

(51) Int. Cl. C12Q 1/68; C12N 1/08

(52) U.S. Cl. 435/6; 435/270

(57) ABSTRACT

The invention relates to an improved method for the accumulation and stabilization of DNA-containing components from biological materials, especially from blood samples. The DNA-containing sample materials are partially lysed in a lysis-binding buffer system, and the DNA-containing components, such as cell nuclei, are bound to a functionalized solid surface. The system comprises lysis reagents and solid adsorbents, the surfaces of the adsorbents being functionalized with polymers of polymerizable acids or derivatives thereof, to which the DNA-containing components bind. Organic or inorganic solid materials can be used as functionalizable carrier materials.

The invention relates to an improved method for the accumulation and stabilization of DNA-containing components from biological materials, especially from blood samples. The DNA-containing sample materials are partially lysed in a lysis-binding buffer system, and the DNA-containing components, such as cell nuclei, are bound to a functionalized solid surface. The system comprises lysis reagents and solid adsorbents, the surfaces of the adsorbents being functionalized with polymers of polymerizable acids or derivatives thereof, to which the DNA-containing components bind. Organic or inorganic solid materials can be used as functionalizable carrier materials. The remaining components of the sample material are removed. Thereafter, the bound DNA-containing components of the sample material can be further purified, and the DNA is isolated according to well-known techniques. If necessary, the DNA-containing components of the sample are removed from the surface under a specific ionic strength, but further treatment can also be effected by directly using the solid phase-bound DNA-containing components. In a specific embodiment of the invention the solid adsorbents have magnetic properties and/or exhibit the form of microparticles with a diameter ranging from 1-100 μm.

With the establishment of DNA-analytic methods in laboratory practice, especially in clinical analytics, methods of purifying nucleic acids have gone through a rapid technological development. Special attention is drawn particularly by methods of isolating nucleic acids, which are suitable for use on automatic “liquid handling” systems. In this context, concepts of solid-phase extraction utilizing adsorbents with magnetic properties are outstanding because manual interventions in the course of extraction are evaded as a result of possible manipulation of these adsorbents with magnetic fields, so that the process can be made a fully automatic one.

It is precisely in clinical analytics, e.g. investigation of the major histocompatibility complex (MHC, HLA analytics), where a specific minimum quantity and concentration of DNA is expected following isolation which requires the use of relatively large amounts of sample material. However, automatic extraction of such relatively large amounts of samples represents a problem because the pipetting robots available are not capable of effectively processing the required volumes in any case. In particular, this applies to the isolation of DNA from blood samples because, as is well known, the cellular proportion of blood largely consists of red blood cells (erythrocytes). Lacking a nucleus, however, they are unsuitable for DNA isolation, so that only white blood cells, i.e. leukocytes, are of interest.

In addition, problems with space frequently arise when storing blood samples of large volumes. Inter alia, this is currently managed by storing so-called buffy coats, the production of which, however, can only be effected manually. Due to high cell concentrations, the subsequent isolation of DNA from buffy coats can be difficult. Another problem is the instability of DNA-containing components immediately after sampling. Whole blood and buffy coats must be stored in a cool place in order to prevent DNA degradation.

Accordingly, strategies have been developed, involving accumulation of the DNA-containing cells or DNA-containing components of the sample material, especially of blood, prior to the actual DNA extraction.

Such procedures envisage several operation steps. Cells from which DNA is to be isolated are accumulated e.g. by means of centrifugation, subsequently lysed, centrifuged once more, followed by contacting the lysate with specific carriers which bind the DNA. One of these variants of accumulating DNA-containing components consists in the chemical lysis of cells by means of so-called lysis buffers for red blood cells (red cell lysis buffer, RCB), pelletizing the DNA-containing components of the blood by centrifugation and subsequent extraction of the DNA from this pellet [Epplen & Lubjuhn (1999), DNA profiling and DNA fingerprinting, Birkhauser Verlag, Berlin, p. 55].

The above methods lack automation compatibility to some extent because the centrifugation steps impede continuously automated extraction of nucleic acids. While integration of centrifuges in robot systems is possible, the implementation is exceedingly cost-intensive and difficult to carry out in technological terms.

Another methodical approach utilizes the affinity of specific antibodies to DNA-containing blood cells (e.g. CD4 cells). These antibodies, bound to magnetic particles, allow concentration of DNA-containing blood cells and, as a consequence, reduction of the volume relevant to the actual DNA isolation. One variant of isolating DNA-containing blood cells by means of specific antibodies bound to magnetic particles has been described by Hardingham et al. [Hardingham et al. (1993), Cancer Research 53, 3455-3458; Lundeberg & Larsen (1995), Biotechnology Annual Review 1, 373-401]. Disadvantages of the above-mentioned method are, firstly, the high price of the magnetic particles being employed and, secondly, the fact that this method fails when using frozen blood samples, as is quite common in clinical practice, because freezing and thawing of the sample material destroys the cells, and the specificity of the antibodies is no longer effective.

The invention is therefore based on the object of developing a method for the accumulation and stabilization of DNA-containing components from biological materials, especially blood, said method being intended to provide materials allowing subsequent easy and fully automatic isolation of DNA and avoiding disadvantageous large sample volumes and centrifugation steps interfering with DNA isolation. Furthermore, stable storage of the DNA in the sample material should be possible under favorable temperature conditions.

According to the invention, said object is accomplished by subjecting the biological materials in a first step to partial lysis in the presence of at least one lysis reagent and at least one solid adsorbent having a surface functionalized with polymers. The DNA-containing components simultaneously bind to the solid surface which, according to the invention, consists of polymers comprising a carrier polymer, preferably of polymerizable acids or derivatives of polymerizable acids, or of polymers comprising a mixture of the above-mentioned carrier polymer and other polymerizable acids or derivatives thereof, preferably selected from sulfonic acid, phosphonic acid or carboxylic acid. Other polymerizable acids or derivatives thereof comprise those polymerizable acids or derivatives thereof which, in a special embodiment of the solid surface, are not identical with those of the carrier material and will also be referred to as acid component in the present invention. Following removal of the supernatant, the bound DNA-containing components are subjected to DNA isolation according to per se known methods. To this end, they can be eluted first.

Surprisingly, it was determined that it is not the free DNA that binds to the functionalized surfaces of the solid adsorbent, but DNA-containing components of a lysate which includes large amounts of intact components of the cytoplasm, especially cell nuclei such as leukocyte nuclei of blood.

The DNA-containing components of the sample material, fixed to the adsorbents according to the invention, are protected from degrading enzymatic or chemical influences and stable, and for this reason, they can be stored or transported in an uncomplicated way, especially at room temperature.

In particular, the method according to the invention facilitates the isolation of DNA from large sample volumes, because the originally large volumes, e.g. from blood samples, are reduced to the DNA-containing components. The possibility of storing sample materials from which DNA is to be isolated subsequently is immensely improved.

The surface polymers preferably consist of carrier polymers of acrylic acid or methacrylic acid or derivatives thereof, such as acrylamide, methacrylamide or acrylic esters.

In addition, the polymer surfaces may include polymerized acids as second component, preferably sulfonic, phosphonic or carboxylic acids, or polymerized derivatives of polymerizable acids, preferably sulfonic or phosphonic acid compounds, more preferably vinylphosphonic acid, vinylsulfonic acid or derivatives thereof, such as styrenesulfonic acid. Preferred in the meaning of the invention are copolymers of a carrier polymer and acid component, i.e. sulfonic acid or vinylsulfonic acid.

Optionally, other monomer components having a polymerizable double bond, such as vinyl acetate, silyl compounds containing vinyl groups, and vinyl stearate, can be used. Especially valuable is the use of the latter monomers to achieve specific surface properties of the adsorbent, such as wettability or modifiability.

In the event of copolymers, the surface polymers are composed with a defined ratio of the different monomers, in binary systems preferably at a ratio of from 9:1 to 1:1 of carrier polymer to acid component, more preferably at a ratio of from 9:1 to 3:1.

The content of the polymerizable acid component in the reaction mixture is between 10% w/w and 50% w/w, preferably between 10% w/w and 25% w/w. In a preferred fashion the functionalized surfaces have styrenesulfonic acid with a weight percentage between 10% w/w and 50% w/w, more preferably between 10% w/w and 25% w/w.

The carrier materials for the polymers of the invention can be any inorganic or organic materials allowing activation as a result of their chemical properties. Likewise, inorganic or organic materials can be utilized, which can be embedded in the polymers according to the invention e.g. by crosslinking soluble derivatives of polymers. Examples of the above include polystyrene, polysulfones, non-modified or modified silica gels. Particularly suitable are polymers bearing hydroxy groups such as cellulose, and especially suited are polyvinyl alcohol derivatives. Furthermore, polyesters, polyamides, polycarbonates etc. can be employed.

Use as carrier materials of the polymers determining the surface properties of the adsorbents is also possible, provided the physical-chemical properties of these materials allow handling in aqueous solutions. In a preferred variant of the method, the polymers coated on the adsorbent surfaces consist of said carrier material and/or vinylsulfonic acid monomers incorporated in the lysed biological material.

In another distinctive embodiment of the invention the solid carrier materials for the accumulation of DNA-containing components consist of microparticles with magnetic properties allowing mechanical manipulation thereof by applying an external magnetic field. In a particularly preferred fashion, micro-particles with magnetic properties and a diameter ranging from 1 to 100 μm, preferably 1 to 30 μm, and more preferably 3 to 10 μm, are employed. Such microparticles are well-known to those skilled in the art. Their production proceeds according to per se known methods, e.g. as described in DE 43 07 262 and U.S. Pat. No. 5,648,124.

The production of the adsorbents required for the accumulation according to the invention can be effected using e.g. graft polymerization processes well-known to those skilled in the art, such as coating the monomer mixtures on surfaces activated by means of peroxide free radicals.

Thus, for example, polyvinyl alcohol derivatives crosslinked with dialdehydes can be activated using a concentrated solution of hydrogen peroxide [Bates & Shanks (1980), J. Macromol. Science Chem. A14, 137-151; Bolto et al. (1978), J. Appl. Polym. Sci. 2, 1977]. Activation of the base surface using partial oxidation with cerium(IV) ammonium sulfate is also conceivable [Mukopadhyay et al. (1969), J. Polym. Sci. A-17, 2079]. Other activation methods are photochemical activation of the surface using sensitizers such as benzophenone or methylene blue.

Furthermore, chemical binding of the polymers determining the surface properties of the adsorbents is possible via so-called anchoring groups situated on the solid carrier materials. Thus, for example, the polymers can be condensed to amino groups situated on the surface of the carrier materials. A person skilled in the art will know how to place amino groups on the carrier materials.

Crosslinking of soluble derivatives of the polymers according to the invention using suitable crosslinking reagents in the presence of the solid organic or inorganic carrier materials also furnishes adsorbents having the properties according to the invention.

Furthermore, the surface properties of the adsorbents can be influenced through the additional use of further monomer components having a polymerizable double bond. Thus, for example, the wetting behavior of the adsorbents can be improved by using vinyl acetate and hydrolysis thereof following polymerization.

The solid adsorbents can preferably be used as a loose powder or as a filter material which can be modified. Particularly preferred is the use as filter matrix in filter plates. The use of the above-described adsorbents in so-called spin columns, i.e. small chromatography columns for handling in table centrifuges, may be mentioned as an example.

To accumulate the DNA-containing components, the functionalized adsorbents can be incorporated in the biological material—preferably present in the form of a biological solution—prior to, simultaneously with, or after lysis. Inter alia, that point in time will be determined by the nature of the adsorbents. When using the adsorbents as filter matrix, for example, the biological samples are contacted with the above-described adsorbents preferably after lysis, so that the DNA-containing components can bind to the functionalized surfaces.

As a loose powder, the solid adsorbents are incorporated in the biological materials preferably in the presence of said lysis reagents. The biological material is lysed, and the DNA-containing components of the sample material bind to the functionalized surfaces.

Biological materials in the meaning of the invention can be body fluids such as blood, urine or cerebrospinal fluid. Moreover, plasma, cells, buffy coats, leukocyte fractions, sputum, sperm, or organisms (unicellular organisms such as eukaryotes or prokaryotes, multicellular organisms, insects etc.), for example, can be used as further biological materials. Furthermore, such biological materials may include cultures of microorganisms, cellular materials such as tissues or soil samples, components of plants or other organisms. The method of the invention is particularly suitable for the accumulation of DNA-containing components in blood (human whole blood), buffy coats, leukocyte fractions and cell cultures.

DNA-containing components in the meaning of the invention are preferably cell nuclei and other DNA-containing organelles such as mitochondria, chloroplasts, or DNA-containing protein complexes included in the sample material, but also DNA-containing viruses such as hepatitis C virus, cytomegalovirus, etc.

The lysis reagents possibly give rise to an osmotic shock, opening the cell membranes. Other conditions of lysis interfering with the stability of the cell structure, such as mechanical exposure to ball mill, French press, ultrasound etc., enzymatic degradation of cell walls or cell membranes by cell wall-lytic enzymes and/or exposure to surface-active substances, are also conceivable.

Solutions containing detergents such as Triton X-100, Tween 20, Tween 80, NP-40 and Briej 35 are particularly suitable as lysis reagents to accomplish the object of the invention. The detergents can be used both as a single component and in combination with a complexing agent from the series of chelating ligands and/or together with a native carbohydrate, preferably an oligosaccharide consisting of at least 50% glucose units, more preferably a disaccharide such as saccharose. Furthermore, ionic detergents such as cetyltrimethylammonium bromide (CTAB) or sodium dodecylsulfate (SDS) can be used. By way of micelle formation, the anionic detergent SDS dissolves lipids out of the cellular membrane, the destroyed structure of which provides targets for cellular enzymes to further degrade the cell wall.

Optionally, salts of uni- or bivalent cation and/or cell wall-lytic enzymes, such as glucanases, proteases, cellulases, etc. can be added to the lysis reagent as a single addition or in combination.

According to the invention, lysis reagents are preferably used which include 0.5% v/v to 5% v/v of complexing agent and/or 0.5% v/v to 3% v/v of detergent, with a volume percentage of 1.0% v/v to 1.5% v/v of detergent being preferred. Particularly preferred is a lysis reagent comprising Triton, saccharose and/or ethylenediamine tetraacetate (EDTA). Even more preferred is a reagent containing 0.5 M EDTA, 1% v/v Triton X-100 and 2.5 M saccharose.

The lysis reagent is preferably used in combination with magnetic microparticles comprising a surface functionalized by means of acrylamide, methacrylamide, acrylic acid derivatives and/or polymerizable acids or derivatives thereof, preferably sulfonic acid derivatives, to bind the DNA-containing components.

Surprisingly, binding of DNA-containing components such as cell nuclei, mitochondria, chloroplasts or DNA-containing protein complexes, but also of DNA-containing viruses to the adsorbent proceeds under the conditions of partial lysis as is present when the cell membrane is destroyed. Under such conditions, pure DNA will not bind to the adsorbent and cannot be isolated in exemplary investigations using the above-described buffer systems.

The remaining components of the sample material are removed. In one embodiment of the method according to the invention the adsorbents with bound DNA-containing components are removed from the remaining sample material after a binding period of the DNA-containing components of 1-10 minutes, preferably 2-5 minutes. The DNA-containing components included in the sample material are concentrated correspondingly, so that the volume introduced into a method of DNA isolation can be dramatically reduced. Using the present accumulation method, a reduction in volume to at least ¼ of the original sample, preferably to ⅛, and more preferably to less than 1/10 is achieved. Following binding, the DNA-containing components can be eluted from the adsorbents, if desired. Optional elution of the DNA-containing components can be effected immediately after discarding the lysed sample material or after intermediate storage. To this end, a small volume of an aqueous salt solution having a defined ionic strength is used, for example. In a preferred fashion, alkali halides and alkaline earth halides, such as NaCl, KCl or CaCl₂, more preferably lithium or calcium halides, and even more preferably lithium chloride and calcium chloride, alone or in mixtures with each other, are used as salts. The salts can either be used as the only components in aqueous solution or as components of aqueous buffer solutions with other components, e.g. detergents or complexing agents known to those skilled in the art, at a preferred concentration of from 0.01 M to 3.5 M, preferably at a concentration of from 0.01 to 1.0 M.

When using the method according to the invention, the volume of the solution required to detach the DNA-containing components is significantly smaller than the original volume of the biological sample, so that concentration of the DNA to be purified is achieved and extraction can be effected fully automatically in small volumes, using robots, for example.

Following optional storage and elution, the DNA-containing components thus bound can be subjected to a per se known purification and DNA isolation, which can also be performed in a fully automatic fashion. These procedures are well-known to those skilled in the art. For example, following removal of proteins present in the sample, e.g. by means of phenol/chloroform extraction, the DNA to be isolated can be precipitated by adding salts, e.g. sodium acetate, or by adding an organic solvent, especially an alcohol such as ethanol or isopropanol, or can be further purified using a well-known solid-phase extraction principle such as binding of DNA to silicate materials in the presence of chaotropic substances. Purification can also be effected by means of gel filtration, gel elution or by using ion exchangers. It is possible to combine a plurality of the methods mentioned above.

The accumulation step according to the invention can be integrated into a fully automatic procedure, because it is only the technical configuration of the adsorbent being used that determines which automatic procedure is used in the accumulation of DNA-containing components.

Without intending to be limiting, the invention will be explained in more detail with reference to the following examples.

### EXAMPLE 1

Production of Functionalized Magnetic Microparticles

1 g of magnetic microparticles consisting of polyvinyl alcohol crosslinked with glutardialdehyde [Bolto et al. (1978), J. Appl. Polym. Sci. 2, 1977] is activated with 10 ml of 30% hydrogen peroxide in the course of one hour. The particles are removed from the suspension and washed peroxide-free with water. Thereafter, the particles are added to a solution of 1.1 g of acrylamide and 0.5 g of styrenesulfonic acid adjusted to pH 7 with sodium hydroxide solution. Following addition of 40 mg of ferrous sulfate, this is stirred for one 1 hour at room temperature. Next, the particles are sucked off, made free of residual monomer and non-grafted polymer using water, and are ready for use in the accumulation of DNA-containing components.

### EXAMPLE 2a

Accumulation of DNA-Containing Components

The magnetic particles produced in Example 1 (10 mg) are placed in a mixture of 2 ml of blood and 4 ml of a lysis buffer consisting of a 2.5 M solution of saccharose containing 1% v/v Triton X-100. The solution is mixed thoroughly to dis-perse the magnetic particles and incubated for 10 minutes at room temperature. Thereafter, the particles are removed by placing a permanent magnet on the vessel wall, and the liquid in the vessel is discarded, taking care not to lose any magnetic particles.

Next, the particles having the bound DNA-containing components are resuspended in 200 μl of 1.5 M NaCl solution. Again, the magnetic particles are collected by applying an external magnetic field at the vessel wall. The supernatant can now be used in further methods of DNA purification.

### EXAMPLE 2b

Accumulation of DNA-Containing Components

The magnetic particles produced in Example 1 (10 mg) are placed in a mixture of 2 ml of blood and 4 ml of a lysis buffer consisting of a 2.5 M solution of saccharose containing 1% v/v Triton X-100 and 0.5 M EDTA. The solution is mixed thoroughly to disperse the magnetic particles and incubated for 10 minutes at room temperature. Thereafter, the particles are removed by placing a permanent magnet on the vessel wall, and the liquid in the vessel is discarded, taking care not to lose any magnetic particles.

Next, the particles having the bound DNA-containing components are resuspended in 200 μl of 1.5 M NaCl solution. Again, the magnetic particles are collected by applying an external magnetic field at the vessel wall. The supernatant can now be used in further methods of DNA purification.

### EXAMPLE 3

Storage of the Accumulated DNA-Containing Components

Accumulation proceeds as described in Example 2. Following removal of the complex of DNA-containing components and particles from the supernatant by applying a permanent magnet, this complex is stored for one week at a temperature from room temperature to 30° C. and is subsequently used in DNA isolation. The amount of isolated DNA and the quality thereof correspond to the order of magnitude of those parameters achieved in comparative instantaneous extraction immediately after accumulation.

Further storage of a complex for another week at 4° C. prior to DNA isolation shows the same results with respect to amount and quality of the DNA.

1-18. (canceled)

19. A method for the accumulation and stabilization of DNA-containing components, characterized in that DNA-containing sample material is partially lysed in a lysis-binding buffer system comprising at least one lysis reagent and at least one solid adsorbent to release DNA-containing components, and the DNA-containing components are bound to the adsorbent, the surface of the adsorbent being functionalized with polymers consisting of a carrier polymer and/or acid component(s) of polymerizable acids or derivatives of polymerizable acids selected from acrylic acids or methacrylic acids, acrylamides or methacrylamides or acrylic esters, or from copolymers of carrier polymer and an acid component, the latter being selected from sulfonic acids, phosphonic acids or carboxylic acids.

20. The method according to claim 19, characterized in that a component not containing DNA is removed from the adsorbent in an additional step.

21. The method according to claim 19, characterized in that the adsorbent is added to a solution of the biological DNA-containing sample material prior to, simultaneously with, or after the lysis step.

22. The method according to claim 19, characterized in that copolymers with a monomer ratio of from 9:1 to 1:1, preferably from 9:1 to 3:1, of carrier polymer to acid component are used.

23. The method according to claim 19, characterized in that the content of the acid component in the reaction mixture is between 10% w/w and 50% w/w, preferably between 10% w/w and 25% w/w.

24. The method according to claim 19, characterized in that a vinylsulfonic acid derivative, preferably styrenesulfonic acid, is used as the acid component.

25. The method according to claim 19, characterized in that the adsorbent consists of organic or inorganic solid carrier materials to which the polymers are bound, preferably polystyrene, polysulfones, modified or non-modified silica gels, polyesters, polycarbonates, polyamides, or polymers bearing hydroxy groups, preferably cellulose, or polyvinyl alcohol derivatives.

26. The method according to claim 25, characterized in that microparticles with an average diameter of 1-100 μm, preferably 1-30 μm, are used as the adsorbent.

27. The method according to claim 25, characterized in that magnetic microparticles are used as the adsorbent.

28. The method according to claim 19, characterized in that the lysis reagent comprises at least one detergent in mixture with at least one native carbohydrate, preferably an oligosaccharide, and/or at least one complexing agent.

29. The method according to claim 28, characterized in that a non-ionic detergent is used, preferably derivatives of the Triton, Tween, NP-40 series or mixtures thereof.

30. The method according to claim 28, characterized in that a disaccharide, preferably saccharose, is used as the oligosaccharide.

31. The method according to claim 28, characterized in that EDTA is used as complexing agent.

32. The method according to claim 28, characterized in that the lysis reagent comprises Triton X-100, preferably 1% v/v, saccharose, preferably 2.5 M, and/or EDTA, preferably 0.5 M.

33. The method according to claim 19, characterized in that biological material is used as DNA-containing sample material, preferably blood, leukocyte fractions, buffy coats, urine, serum, plasma, cell suspensions of microorganisms, or digested material of plants.

34. The method according to claim 19, characterized in that cell organelles, preferably cell nuclei, mitochondria or chloroplasts, DNA-containing protein complexes, or DNA-containing viruses are accumulated as DNA-containing components.

35. The method according to claim 19, characterized in that the DNA-containing components are removed from the solid adsorbent using aqueous salt solutions preferably containing alkali and alkaline earth halides.

36. The method according to claim 35, characterized in that alkali and/or alkaline earth chlorides, preferably lithium and/or calcium chloride, are used at a concentration of from 0.01 to 3.0 M, preferably at a concentration of from 0.01 to 1.5 M.
